(19) United States
(12) Patent Application Publication
(10) Pub. No.: US 2008/0131487 A1
(43) Pub. Date: Jun. 5, 2008

(54) CHYMOSIN FOR THE PREVENTION AND TREATMENT OF GASTROINTESTINAL DISORDERS

(76) Inventors: Dag Ommundsen, Fevik (NO); Havard Skjarvik, Farvik (NO)

Correspondence Address: JAMES REMENICK;NOVAK DRUCE & QUIGG, LLP, 1300 I STREET NW, SUITE 1000 WEST TOWER, WASHINGTON, DC, 20005, US

(21) Appl. No.: 11/839,960

(22) Filed: Aug. 16, 2007

Related U.S. Application Data
(60) Provisional application No. 60/861,728, filed on Nov. 30, 2006.

(51) Int. Cl. A61K 38/48; A61K 47/00; A61P 1/00

(52) U.S. Cl. 424/439; 424/94.66

(57) ABSTRACT

The present invention is directed to medical or dietary treatments comprising administration of a zymogen such as chymosin or other rennins. Embodiments of the invention are directed to treating infant colic, heartburn, gastro-esophageal reflux (GER), gastro-esophageal reflux disease (GERD), irritable bowel syndrome, and recurrent abdominal pain. The invention is also directed to improving absorption of calcium from milk for osteoporosis patients. Further the present invention may have a very positive effect on life quality for groups with different chronic digestion problems such as ulcerous colitis, multiple sclerosis, migraines, weight disorders and diabetes. Infants may be given several drops of a composition containing chymosin prior to every feeding, regardless of whether they are bottle or breast-fed. The composition containing chymosin can also be incorporated into a food product such as drinking milk or baby formula. The rennin helps infants break down milk proteins so that the milk can be transformed into cheese pulp in the stomach, mimicking the process occurring in the stomachs of normal infants.

### REFERENCE TO RELATED APPLICATIONS

This application claims priority to U.S. Provisional Application No. 60/861,728, entitled “Chymosin Treatment for Infant Colic” filed Nov. 30, 2006, which is specifically and entirely incorporated by reference.

### BACKGROUND

1. Field of the Invention

This invention is directed to pharmaceutical compositions and methods for the treatment and prevention of gastrointestinal and other disorders in humans and other mammals such as, for example, newborns, infants, toddlers, children, teens, adults and seniors. In particular, treatments involve administering a zymogen that remains inactive, and thus stable, until activated by the natural action of administration or deliberate action. A typical zymogen is rennin such as chymosin, which can be used to treat or prevent, for example, infantile colic, heartburn, gastro-esophageal reflux (GER), gastro-esophageal reflux disease (GERD), irritable bowel syndrome and recurrent abdominal pain.

2. Description of the Background

2.1 Infantile Colic

Colic is a common and distressing condition in babies, characterized by excessive and apparently unjustified crying. Although it occurs in approximately 20% of infants starting at a few weeks after birth and improving by about the fourth month, it remains unexplained and untreatable by the medical community. Colic can therefore be very stressful for parents who find themselves unable to comfort their crying babies, despite the fact that the condition is not permanent or harmful to the child, who continues to develop normally.

The symptoms of colic include recurring intensive crying episodes, usually in the late afternoon or evening, lasting from a few minutes to several hours. Crying episodes may be followed by a bowel movement or the passing of gas. Additionally, babies with colic may draw their legs towards their abdomens, clench their fists, tense their abdominal muscles, and thrash about appearing in pain. Because of the vigorous and unyielding crying, babies may also become red in the face.

The causes of colic remain largely unknown, although several possibilities have been put forth by researchers, including cow milk protein intolerance, lactose intolerance, immature digestive systems, poor digestion, the backing up of food into the esophagus, intestinal gas, hormonal changes, and the diet of breast-feeding mothers. Diagnosis of colic is based on the pattern of behavior exhibited by a baby, and the elimination of other possible physical explanations, such as urinary infections, intestinal obstructions or other injuries or medical problems.

Similarly to the causes of colic, colic medications also remain unknown, with treatments mainly focusing on symptom reduction (I Jakobsson, L Lothe, D Ley and MW Boschel, Effectiveness of Casein Hydrolase Feedings in Infants with Colic, Acta Paediatr 89:18-21.2000). Several studies have suggested the use of digestive enzymes as supplements for the treatment of colic, aiding in the digestion of milk (K Padiatelli, K. Konstantopoulou & D. Tsitsilianou, Treatment of Infantile Colic in the First Three Months of Life with the Use of a Polyvalent Preparation of Digestive Enzymes, From the Pediatrics Department of the Maternity Hospital “Marika Iliadi”; Patrea L. Pabst, Enzyme Supplemented Baby Formula, U.S. Pat. No. 5,902,617). Some prescription medications which may be used to treat the symptoms of colic can have serious side effects. Lactase, a food supplement consisting of the enzyme lactase, can be used to help break down lactose for lactose-intolerant babies. Simeticone, an anti-flatulent, can also be used to reduce gas. However, it has not been proved that lactose-intolerance causes colic, and gas is merely one of a series of symptoms in colicky babies. Moreover, pharmaceutical treatments may not always be safe for small babies, as even bicarbonate-containing antacids or herbal teas may be dangerous to the baby. The only possible courses of action being promulgated for safely alleviating the symptoms of colic remain basic care and tenderness from the parents including holding and rocking the baby, changes in diet of the breast-feeding mother (which has not been proven to be effective), changes in feeding schedules, use of a constant background noise (i.e. white noise), changes in the shape or holes of bottle teats, and the use of pacifiers. Alternative medicines such as chamomile and peppermint tea, chiropractic and massage therapy, and acupuncture have also not been proven to be helpful or safe. Furthermore, the Food and Drug Administration has issued an advisory warning against star anise tea as a treatment for colic because researchers have discovered low levels of veranisatin, a toxic compound, in the tea, and have found that the tea may cause irritability, vomiting, and even neurological symptoms such as seizures.

2.2 Heartburn, Gastro-Esophageal Reflux (Disease) (GER, GERD)

Heartburn is a painful burning feeling in the chest and sometimes in the throat. Heartburn is extremely common in the United States and Europe. It is one of the most common reasons why people go to gastroenterologists or their family doctors. About 25% of the population takes antacids at least once or twice a month, and 7 percent of the population experiences heartburn every day. The major cause of heartburn is gastro-esophageal reflux (disease). Gastro-esophageal reflux disease (GERD) is present when the passage of gastric contents into the esophagus causes damage in the mucosa. GERD is defined by the presence of symptoms or complications from GER (gastro-esophageal reflux), and GER can be a normal physiologic process in many infants. The frequency or prevalence of GER can be of 50% in infants of ages 0 to 3 months, decreasing to 5% in infants of 12 months. Thus, the majority of infants “outgrow” their reflux. However, determining whether reflux is physiological or pathological can be a major challenge.

GERD is also a common cause of a variety of symptoms and is associated with a number of significant diseases. In adults and children with asthma there is increasing evidence that 60-90% of patients have gastro-esophageal reflux, and a large number of these patients require interventions and treatment for their reflux in order to control their asthma. Reflux is the major cause in 20% of patients with chronic cough, and it is an important cause of recurrent bacterial lung infections. There is also increasing evidence that there is an important link between “high” GERD and disorders in the upper airways such as hoarseness and vocal cord pathology, chronic sinus disease, chronic middle ear disorders with effusions, and apnea in infants/ALTE (Apparent Life-Threatening Events). Long term duration of reflux increases the risk for permanent damage and scarring in the esophagus, and recent publications confirm that there is an increased risk of esophageal adenocarcinoma. Persistent GERD symptoms from infancy to childhood are an important predictor of chronic symptoms in adult life. This underlines the importance of early diagnosis and early and successful treatment.

The causes of gastro-esophageal reflux are only partially understood, and the failings and general penury of today's treatment options understate this. Among the possible causes are genetic influences with familial clustering as well as lifestyle factors such as smoking, obesity and dietary behavior. The structure and function of the gastro-esophageal junction (the passage between the esophagus and the stomach) is of key importance in reflux disease, as the condition becomes more severe with increasing refluxate during transient relaxations of the lower esophageal sphincter. In some patients, a factor may also be an abnormal and delayed passage of content through the stomach and into the duodenum. Gastric transit time is probably delayed in a percentage of patients with GERD, and in these patients, thickening of food or drugs with prokinetic effects may help to resolve the problem. Measuring the ph in the esophagus and gastroscopies with biopsy results have shown that a significant number of patients with GERD have acid stomach content passed into the lower and upper esophagus. A larger number of these patients are successfully treated with acid suppressant medications such as H₂ receptor antagonists and proton pump inhibitors. A majority of the population with these symptoms do not have adequate contact with the health care system. This results in an overuse of antacids which are good for intermittent relief but are not curative and give unacceptable side effects especially in children.

The lack of effect of acid suppressive therapy as well as newer esophageal investigations show that at very large number of patients with suspected GERD have non-acid reflux. The symptoms may be the same, but acid-suppressive therapy would obviously not be appropriate for non-acid reflux. In these patients, prokinetic drugs have previously been used but with mostly disappointing results. Today, however, these therapy alternatives are also no longer used because of serious side effects, among which are cardiac arhythmias. Dietary changes such as avoidance of large meals, caffeine, and spicy foods, to name a few, have limited effect.

2.3 Recurrent Abdominal Pain

Recurrent abdominal pain (RAP) is defined as three or more episodes of abdominal pain influencing daily activity during a period of at least three months. Several epidemiological studies have shown a prevalence of 10% in school age children. The prevalence increases to a maximum in children who are 9 years of age, and then decreases in children older than that. The prevalence is higher in girls. An organic cause is found in 10-25% of cases, in which GERD, constipation and lactose intolerance are the most frequent diagnoses. Non-organic functional abdominal pain is used as diagnosis when no objective organic causes are found. There is an international classification system (the Roma II-criteria) in use to describe the symptoms of patients in which irritable bowel syndrome is one of the diseases.

2.4 Irritable Bowel Syndrome (IBS)

Irritable bowel syndrome is a chronic, episodic functional gastrointestinal (GI) disorder characterized by abdominal pain/discomfort and altered bowel function (constipation, diarrhea, or alternating periods of both). The overall prevalence of IBS in the US is 14.1%, with only 3.3% being medically diagnosed. This compares with a prevalence rate of 11.5% in a previous European study, and is consistent with other large U.S.-based epidemiological studies, where prevalence estimates cluster around 10-15%. Current IBS sufferers (both medically diagnosed and not medically diagnosed) were more likely to have suffered from other GI disorders (previously diagnosed by a doctor) compared with non-IBS sufferers. IBS sufferers were more than twice as likely (22% vs. 10%) to suffer from gastro-esophageal reflux disease (GERD) compared to non-IBS sufferers. Most (76%) irritable bowel syndrome sufferers in the US and Europe are undiagnosed, and the causes of this disorder are largely unknown. Irritable bowel syndrome has a substantial impact on sufferers' well-being and health, with considerable socioeconomic consequences.

2.5 Osteoporosis, Multiple Sclerosis, Ulcerous Colitis, Migraines, Weight-Loss, Diabetes and Milk Lactose and Protein Intolerance

Different studies during the last years have proven that drinking ordinary milk has no major effect on preventing and/or curing osteoporosis. There are reasons to believe that the cause of this disappointing fact may be that the calcium in the milk, in addition to other vitamins, are bonded in the kappa protein, inaccessible by the body. Studies and experience also indicate that a balanced stomach milieu may have a positive effect on combating migraines.

### SUMMARY OF THE INVENTION

The present invention overcomes problems and disadvantages associated with current new compositions, methods, strategies, preventions and treatments for a variety of disorders including, but not limited to infant colic, esophageal disorders, gastro-intestinal disorders, calcium insufficiency and poor blood sugar metabolism, heartburn, gastrointestinal reflux-related symptoms, recurrent abdominal pain, irritable bowel syndrome, insufficient calcium absorption and digestion, and poor blood sugar metabolism. Among the advantages of the current invention is that embodiments of the treatment are simple to manufacture and use, as well as harmless and cost-effective when used properly.

One embodiment of the invention is directed to pharmaceutical and/or nutritional compositions comprising a therapeutically effective amount of a zymogen. Zymogens such as, for example chymosin or another rennin (activated from prorenin), pepsin (activated from propepsin), phospholipase A (transformed from inactive prophospholipase A by tryptic hydrolysis of the Arg-Ma bond), pancreatic enzymes (activated from inactive enzymes), trypsinogen (activated from protrypsinogen), elastase (activated from proelastase), coagulation enzymes, and chymotrypsinogen A (activated from prochymotrypsinogen A), are formulated for administration to patients, including, for example, infants, toddlers, children, adults or seniors. Compositions include forms for direct administration and concentrated forms for dilution into food products such as, for example milk, yogurt, cheese, juice, beer, wine, edible alcohols (e.g. ethanol), cereal, formula, water, or edible oils (e.g. linseed, castor, corn, olive, fish, animal) for subsequent administration (whether therapeutic, prophylactic or nutritional).

Another embodiment is directed to pharmaceutical and/or nutritional compositions and methods comprising a therapeutically effective amount of chymosin or another rennin formulated for administration to patients in a food product. Preferably the composition contains from 1-5,000 IMCU per liter, more preferably from 10-2,000 IMCU per liter, more preferably from 20-500 IMCU per liter, more preferably from 40-200 IMCU per liter, more preferably from 5-200 IMCU per liter, more preferably from 10-400 IMCU per liter, more preferably from 20-250 IMCU per liter, and more preferably from 10-50 IMCU per liter. Concentrated forms can be created for ease of storage and transportation, to be diluted before administration and/or use.

Another embodiment is directed to pharmaceutical and/or nutritional compositions of the invention wherein the enzyme has an activity of, or inactive enzyme which when activated would have an activity of 1-30,000 IMCU/ml, more preferably 1,100-30,000 IMCU/ml, more preferably 1-1,100 IMCU/ml, more preferably 500-1,100 IMCU/ml, more preferably 200-500 IMCU/ml, more preferably 100-200 IMCU/ml, more preferably 1-100 IMCU/ml, more preferably 50-100 IMCU/ml, more preferably 1-50 IMCU/ml, and more preferably 10-20 IMCU/ml. Compositions may comprise therapeutically effective amounts of enzyme or be in more concentrated forms, either as dry material or liquid suspension that is diluted in carrier or a food substance before use.

In certain embodiments, the therapeutically effective amount of enzyme varies according to how rapid a reaction is desired, the weight of the patient, or the amount or consistency of a food product ingested by the patient, all of which can be empirically determined by those skilled in the art.

Another embodiment is directed to pharmaceutical and/or nutritional compositions and methods directed to treating, preventing, or ameliorating the symptoms of infant colic, heartburn, gastro-esophageal reflux (GER), gastro-esophageal reflux disease (GERD), irritable bowel syndrome, recurrent abdominal pain, osteoporosis, obesity, weight problems, ulcerous colitis symptoms, chronic digestion problems, multiple sclerosis or symptoms of multiple sclerosis, diabetes, migraines, digestive disorders, stomach disorders, bowel disorders, or combinations thereof.

Another embodiment is directed to methods for treating gastrointestinal and other disorders by administration of compositions of the invention. Preferably, inactive rennin is administered orally to the patient and the enzyme is activated upon contact with the patient's gastrointestinal tract. Alternatively, inactive rennin can be activated by heat and/or a decreased or more acid pH prior to administration. Administration can be to newborns, infants, toddlers, children, teens or adults as needed or desired to relieve or prevent gastrointestinal and/or other disorders, including associated symptoms allowing the disorder to resolve naturally or with time. Compositions are preferably administered orally, which can be before feeding, during feeding, after feeding, or a combination thereof.

Another embodiment is directed to compositions and methods that promote calcium absorption by the gastrointestinal tract of the patient.

Another embodiment is directed to compositions and methods that act by enzymatically digesting or breaking down milk proteins and coagulating milk inside a stomach.

Another embodiment of the invention is directed to compositions and methods comprising administration of chymosin and/or another rennin, wherein the composition is added to a food product. In another embodiment, the food product is drinking milk or infant formula. In another embodiment, the compositions are in powder or liquid form.

Other embodiments are directed to a method for producing the compositions of embodiments of the invention.

Another embodiment is directed to the administration of compositions of the various embodiments of this invention to patients who are infants, mammals, or humans.

Other embodiments and advantages of the invention are set forth in part in the description, which follows, and in part, may be obvious from this description, or may be learned from the practice of the invention.

### DESCRIPTION OF THE INVENTION

Colic is a distressing condition in newborns. In mild cases, it is typically characterized by excessive and continued crying after mild feeding. The main period of susceptibility starts shortly after birth through to about the fourth or fifth month and sometimes longer. Crying episodes last from minutes to hours and are typically followed by bowel movements or the passing of gas.

It was surprisingly discovered that the symptoms of colic in an infant could be completely or nearly completely alleviated by administering a small amount of active chymosin. In particular, it was found to be sufficient to apply a small quantity of chymosin to the lips in liquid form while bottle feeding milk or infant formula. Through natural means such as simply swallowing and feeding, the chymosin is able to reach the gastrointestinal track and alleviate and even prevent, when administered immediately or shortly prior to feeding, symptoms associated with colic. Chymosin causes cleavage of the peptide bond between the phenylalanine and methionine. With regard to milk protein or casein, the bond between para-casein and the acidic glycopeptide group of casein is between a phenylalanine and methionine and therefore the principal target for chymosin. The resultant product of cleavage is calcium phosphocaseinate, which does not cause bloating or gas production in the stomach, and thus, no symptoms associated with colic.

Native chymosin of animal origin has been commercially available for many years. It is typically isolated by extraction from animal tissues that contain one or several of these types of enzymes. Chymosin is one of a group of enzymes called rennins. Rennins are aspartic endopeptidases having molecular weights in the range of about 35,000 to 42,000 Daltons (group 3.4.23 according to the Enzyme Nomenclature, 1992 of the International Union of Biochemistry and Molecular Biology, IUBMB) such as pepsin A (3.4.23.1) and gastricsin (3.4.23.3) which is excreted into the gastric juice of vertebrates including ruminants, pigs and humans, and chymosin (3.4.23.4) which is a predominantly neonatal gastric enzyme with high milk clotting activity, excreted in mammals. The molecular weights of animal milk clotting enzymes are in the range of about 35,000 to about 42,000 Daltons. Chymosin has a calculated molecular weight of 35,652 Daltons.

In addition to enzymes of animal origin, several natively produced microbial enzymes are used in the dairy industry. These enzymes are generally referred to as microbial milk clotting enzymes or microbial coagulants. Examples of such enzymes include Rhizomucor (previously Mucor)miehei proteases including destabilized, i.e. oxidized Rhizomucor miehei protease, Mucor pusillus protease and Endothia parasitica protease.

As used herein, the term rennin refers to a class of enzymes know as aspartic endopeptidases and includes, but is not limited to pseudochymosin, chymosin, prochymosin, pepsin A, gastricsin, Rhizomucor miehei proteases, Mucor pusillus protease, Mucor pusillus protease, and Endothia parasitica protease, whether isolated and/or purified or recombinantly produced, as well as variations and derivative of these enzymes that retain useful enzymatic function as described herein.

As embodied and broadly described herein, the present invention is directed to compositions containing a rennin such as, for example, chymosin. The composition may contain active enzyme, inactive enzyme (e.g. zymogen) or partially active enzyme that is sufficiently stable to be therapeutically beneficial for administration.

Chymosin is synthesized within the cells of the stomach lining in a precursor form known as preprochymosin. The “pre” portion of preprochymosin is a sequence of amino acids located at the amino terminus. These amino acids comprise a signal peptide which appears to be involved in the transport of the protein to the cell wall for secretion into the periplasmic space. The signal peptide is cleaved at the cell wall and the enzyme is secreted as prochymosin. Prochymosin is a zymogen containing 365 amino acids (40,477 Daltons). Prochymosin is converted to chymosin by the specific removal of 42 amino-terminal amino acids from the enzyme molecule. The conversion of prochymosin to chymosin occurs in a low pH environment and is favored by the low pH environment of the stomach and gastrointestinal tract.

Chymosin has been separated and purified using various techniques. For example, calf rennet or rennet extracts have been purified using blue dye affinity ligands, as described in U.S. Pat. No. 4,666,843 or using a cellulose resin column, as described in U.S. Pat. No. 4,745,063. The same methods have been used to recover and purify microbially produced chymosin, as described in U.S. Pat. No. 4,743,551 and U.S. Pat. No. 4,721,673, respectively. Further, U.S. Pat. Nos. 5,888,966; 5,536,661; 5,215,908; 5,151,358; 5,139,943 and 4,721,673 disclose additional details, methods of isolation and purification as well as variations and derivatives of rennin enzymes. The disclosures of all of these patents are hereby incorporated by reference.

The primary source of rennin enzymes are stomachs of calves and adult cattle in which essentially all of the in vivo milk clotting activity is associated with the presence in the gastric juice of chymosin and pepsin A. When produced in stomach tissue cells, these enzymes occur as the enzymatically inactive pre-enzymes referred to as pre-prochymosin and pre-pepsinogen A, respectively. When chymosin is excreted, an amino-terminal fragment is cleaved off to give prochymosin including a pro-fragment. Prochymosin is an essentially inactive form of the enzyme which, however, under acidic conditions becomes activated to the active chymosin molecule by removal of the pro-fragment. This activation takes place in vivo in the gastric lumen under appropriate pH conditions. Pepsinogen A is activated into the active enzyme by partial hydrolysis under acidic conditions.

Pseudochymosin is the designation of a chymosin species where only part of the pro-fragment or activation peptide (amino acid residues 1-27) is removed. Pseudochymosin will, for example, occur in an extract which has been exposed to a low pH, such as a pH of 2. Pseudochymosin has enzyme activity and is stable at low pH, but is further processed to chymosin at higher pH. Mammalian chymosin can be produced in recombinant microorganisms including filamentous fungi and commercially available. Thus, preferred compositions of the invention contain enzymes isolated and/or purified from traditional or non-traditional sources including microbially produced as well as recombinantly made enzymes

Preparations or compositions containing native milk clotting enzymes of animal origin are prepared industrially by extraction from stomach tissues, in particular from ruminants including calves and adult cattle. Enzyme-containing crude extracts contain chymosin species including precursors, and pepsin species in ratios which depend primarily on the age of the animal. Thus, the distribution between chymosin and pepsin in stomachs from young calves is typically about 80:20 to 90:10 whereas in stomachs from adult cattle it is typically about 25:75. It is understood by those skilled in the art that intermediate distributions between these enzyme species may be found in older calves and young cattle. As an example, the above ratio in extracts from these animals is typically in the order of 50:50. Preferred compositions of the invention contain isolated and purified rennin, but may contain other active or inactive substances that may be carried over from the purification process. Such additional components although active, would not generally affect the intended use of rennin as a pharmaceutical or nutritional supplement of the invention.

Conventional commercial products containing chymosin of animal origin are manufactured by a multistep, time consuming process which typically include: (i) preparing crude enzyme-containing extract by extracting comminuted, frozen or dried calf or cattle stomachs with water, (ii) transforming proenzymes into active enzymes, (iii) a clarification step wherein a flocculant is added to facilitate subsequent filtration, (iv) concentration steps, (v) repeated clarification, (vi) further filtration to remove precipitated impurities, (vii) adjusting salt and preservative concentration, (viii) adjusting the enzymatic strength and composition to obtain the finished product which is usually referred to as rennet. Prior to packaging, the rennet product may be subjected to a final filtration step including a sterile filtration. Conventionally manufactured enzyme preparations contain a mixture of chymosin and pepsin, the latter enzyme being much less active than chymosin in respect to milk clotting.

Enzymes of the invention are typically stored and maintained at a pH above the level whereby inactive proenzyme is converted to active enzyme. In other words, enzymes may be maintained in their inactive state prior to use. At room temperatures and at pH values in the range of 0.5 to 5, pre-enzymes are converted into active endopeptidases. Preferred pH values for conversion are in the range of 1.0 to 3.0, such as in the range of 1.5 to 2.5 including a pH of about 2.0. The lowering of pH is conveniently obtained by the addition to the composition of inactive enzyme of a strong inorganic acid such as H₂SO₄, HCl, H₃PO₄, HNO₃ or an organic acid such as, for example, acetic acid, formic acid or lactic acid. Most preferred are FDA approved acids that do not have to be removed or otherwise isolated from the composition for administration to the patient. Also preferred are ingestible acids (e.g. HCl, acetic acid) that can be neutralized after conversion of the proenzyme with ingestible alkaline compounds such as sodium hydroxide, amines and ammonias or any hydrogen binding compound or salt that is harmless to the patient at the particular dose necessary to sufficiently neutralize the acid. The composition is kept at a low pH for a period of time sufficient to achieve an activation of all or essentially all (meaning at least the therapeutically effective amount) of the enzyme. This activation period is generally in the range of 10 to 120 minutes, preferably in the range of 30 to 60 minutes, more preferably less than 30 minutes and even more preferably less than 10 minutes. It should be noted that a proportion of the activated chymosin is present as pseudochymosin as defined herein, the proportion hereof depending on the pH and the activation process.

The composition of a given batch of animal rennet may vary considerably depending on the animal stomach raw material. When conventional rennet having a relatively high proportion of chymosin is desired, the stomach tissue of from young calves is the preferred raw material. Such products may be designated calf rennet. Rennet products having lower proportions of chymosin may be manufactured on the basis of stomachs from older calves or from adult cattle (ox rennet). Naturally, rennet products having intermediate enzyme chymosin content may be obtained, for example, by mixing a calf rennet and an ox rennet. The strength of a crude extract as described above is typically in the range of 5 to 30 Chr. Hansen units (CHU)/mL. Typically, conventionally manufactured commercial liquid rennet products have an enzymatic strength in the range of 40 to 100 CHUs/mL.

The composition of the invention preferably contains prochymosin and is maintained in its inactive or unconverted state. Compositions are preferably preserved with buffering and stabilizing agents that maintain the composition at a pH above the pH at which prochymosin is converted into chymosin. That pH is preferably above 3, and more preferably above 4, 5, 6, 7 and 8, and at a temperatures at or below room temperature (e.g. 24° C., 20° C., 18° C., 15° C., 12° C., 10° C., 6° C., 4° C., 0° C., −4° C., −20° C.). Any combination of pH, temperature and preserving agent or agents that does not cause premature conversion of prochymosin to chymosin is acceptable. Preferably, compositions of the invention also contain buffering agents, stabilizing agents, anti-oxidants and other preservatives.

Compositions are used for treating or preventing a variety of diseases and disorders such as, for example, infant colic, heartburn, gastro-esophageal reflux (GER), gastro-esophageal reflux disease (GERD), irritable bowel syndrome, and recurrent abdominal pain. The invention is also directed to the improvement of calcium absorption from milk and milk products. Embodiments of this invention are directed to compositions especially formulated for both infants and adults and to methods for making such formulations. In some embodiments of this invention, methods and compositions containing chymosin or another type of rennet are directed at eliminating the need for symptom-reducing treatments including antacid, anti-flatulence, and alternative medicines.

One embodiment of the invention is directed to a pharmaceutical composition comprising chymosin or another rennin formulated with pharmaceutically or nutritionally acceptable carriers for administration to infants, children or adults. Compositions preferably contain a therapeutically effective amount of preferably chymosin in combination with a carrier such as water, saline or another nutritionally or pharmaceutically acceptable carrier for infants.

By adding chymosin to milk, it was surprisingly discovered that milk was provided with a significantly better capability to regulate ph value over prolonged periods of time, including the ability to regulate gastrointestinal motility. These capabilities have a surprisingly positive effect on the quality of life of patients with chronic digestion problems related to disorders such as ulcerous colitis, multiple sclerosis and diabetes.

Embodiments of the invention are also directed towards use in weight loss diets and for diabetic patients. Milk containing chymosin or another rennin, as provided for in embodiments of this invention, creates a feeling of comfortable fullness for a prolonged period of time, preferably for at least 30 minutes, more preferably for between 30 minutes and 1 hour, more preferably for between 1 and 2 hours, even more preferably for at least 2-3 hours, and most preferably for more than 3 hours. At the same time, it was surprisingly discovered that chymosin allows milk to provide a very slow increase in blood sugar.

Embodiments of the invention are also directed towards treating lactose and milk protein intolerance. For example, people who are intolerant to casein protein may drink milk containing chymosin as presented in various embodiments of the present invention, as the chymosin will transform the casein protein into paracasein. This transformation takes place in very few minutes, thus ensuring that the digestive system does not have to process heavy casein proteins. Embodiments of this invention ensure that chymosin provides for a slow and favorable proteolytic phase. Thus, it was surprisingly discovered that chymosin as provided for in embodiments of this invention improves protease in general, as well as the digestion of milk lactose.

Without wishing to be bound by theory, chymosin breaks down milk proteins in patients so that ingested milk can be transformed into cheese pulp in the stomach. Chymosin also affects the acid milieu, enzymatic secretions, and bacterial homeostasis in the stomach and, secondarily, in the lower gastrointestinal tract.

Proper calcium absorption may often be a problem because the calcium in milk is bonded in kappa protein. Embodiments of the invention are directed at splitting the kappa protein, allowing the calcium to be released. The calcium thus becomes more available to the body, together with various vitamins contained in milk. Thus, embodiments of this invention can be used for treating osteoporosis.

The causes of neonatal colic, gastro-esophageal reflux disease as well as recurrent abdominal pain and irritable bowel syndrome are largely unknown. The treatment options of neonatal colic lack, and the treatment options of gastro-esophageal reflux are, in a significant number of patients, limited and often disappointing. It is likely that these diseases have several patho-physiologic factors in common, and milk with added chymosin, as presented in embodiments of the invention, are a surprising and important treatment alternative with no significant side effects. To understand the challenges of treatment, it is essential to understand the function of the stomach, and secondarily, the possibility of functional disturbance of the lower intestinal tract due to persistent dysfunction of the gastric milieu.

The stomach is usually a highly acidic environment due to gastric acid production and secretion which produces a luminal pH range of 1 to 4, depending on food intake and other factors. The stomach's primary function is to break down large molecules (such as from food) into smaller ones using gastric acid so that they can eventually be absorbed from the small intestine. Other important functions include partial protein digestion by pepsin enzymes, and absorbing water, ions, and lipid-soluble compounds. The movement and the flow of chemicals into the stomach are controlled by both the autonomic nervous system and by various digestive system hormones. To maintain this fine gastric environment and regulation, a balance between normal functioning cells in the lining of the stomach (mucosa), gastric secretory enzymes, neurotransmitters, hormones, and other compounds must be maintained. A disturbance of this homeostasis increases the risk for excess or lack of acid, cell damage and a cascade of harmful processes. Primary or secondary gastro-esophageal reflux contributes to an inflammation in the stomach and esophagus. Studies of gastric emptying confirm that gastro-esophageal reflux has a negative impact in some patients. Changes in the gastric milieu is also known to influence colonic transit, as several studies have shown that only one in five non-ulcer dyspepsia patients has normal bowel function based on clinical symptoms; constipation is particularly prevalent.

Milk with chymosin treatments, as embodied herein, optimize the digestive process and balance the pH milieu in the stomach. Secondarily, embodiments of this invention have a positive influence on gastric emptying, reducing the risk of reflux to the esophagus as well as reducing the damage to the lining of the esophagus by buffering the gastric acid. Through a balanced gastric milieu and transit time, absorption in the small and large intestines is greatly improved.

Infant colic continues to be a common and distressing condition in babies. It is typically characterized by excessive and apparently unjustified crying. It has been surprisingly discovered that symptoms can be alleviated by the administration of chymosin, which acts by breaking down milk proteins and cause the milk casein proteins to be transformed into paracasein, while milk is turned into cheese pulp in the stomach. The invention is achieved by targeting the very source for the occurrence of colic in infants. In addition, treatment produces an extended time in which an infant feels full rather than hungry. In colicky infants, milk goes through the stomach in about 15 minutes, after which, the baby feels hungry again. Because digestion of the milk was not complete in the first place, this creates the possibility for undernourishment. Infants who do not have colic, on the other hand, feel full for about two and a half hours after feeding, and they also have better digestion and nourishment.

The present invention is directed towards pinpointing the cause of colic as being an insufficient production of chymosin in the stomach. Chymosin, also known as rennin, aids in breaking down kappa proteins in milk, coagulating it, or transforming it into cheese (Susan R. Kerr, An Overview of Calf Scours, Cooperative Extension Washington State University, Central Washington Animal Agriculture Team, Fact Sheet # 1041-2004; Claire Queguiner, et al., Novel Method for the Production of Fermented Milk Products, WO/03/070009 A1).

With an insufficient amount of chymosin, milk goes through the stomach very quickly, fermenting and creating gases down the colon, and causing the pain and crying experienced by colicky babies. Patients who can be treated include infants, although all other age groups may benefit as well including newborns, toddlers, children, teens, young adults, adults and seniors. In addition, colic is not limited to humans and, accordingly, treatment may be administered to humans, dogs, cats, horses, cows, goats, sheep, and any mammal in need thereof.

The identification of the likely cause of colic was achieved through the observance of what colicky versus normal infants expectorate gas, saliva and other liquids, and also solids such as partially digested food, immediately after feeding. When a normal baby is burped after eating, she expectorates solids that appear as cheese pulp. When an infant who suffers from colic is burped, what is noticed is mainly milk with only a limited amount of cheese pulp in the expectorate.

This invention is directed to a pharmaceutical composition comprised of chymosin or another rennin, which can be orally administered in a therapeutically effective amount to the infant and, preferably, just prior to every feeding, or during, or immediately after, or in a combination of the three. This helps the infant breaks down milk proteins so that the milk can be transformed into cheese pulp in the stomach in the same way that it happens in the stomach of a normal infant. Chymosin can be readily obtained commercially. The enzyme is typically purified from mammalian stomachs (e.g. calf and lamb), but can also be produced recombinantly from microorganisms such as bacteria and yeast, or produced from mammalian cells and cell cultures.

Preferably, an effective amount of chymosin is added to milk, baby formula, or another baby food product, in order to break down casein protein or other milk proteins, preferably allowing the milk to turn into cheese pulp in the stomach. Preferably, this process resembles the natural process of protein break-down in the stomach of non-colicky infants, where chymosin is produced naturally.

Thus, a milk product or milk formula is transformed into cheese pulp in the stomach with the addition of the chymosin of this invention. Embodiments of this invention preferably create a balanced pH value in the stomach, and, over time, this invention will preferably contribute to the building up of good bacterial flora in the stomach. Preferably, digestive system problems are minimized by drinking 1-2, 1-3, or 1-5 glasses or milk containing chymosin per day. A preferred embodiment of this invention optimizes the gastric milieu through the addition of chymosin, and more preferably improves gastric motility, gastric emptying, and bowel function.

Embodiments of this invention are directed to chymosin-containing products which have ameliorative effects on regurgitation and reflux problems, including but not limited to gastritis and heartburn, as well as on digestion problems including irritable bowel syndrome. Abdominal pain in children as well as adults, can also be treated in addition to colic, which often shares the same pathogenesis and treatment challenges as those problems, through the chymosin treatments of this invention.

In other embodiments, chymosin-containing products of this invention may have a positive effect on blood sugar, which allows for the treatment of types I and II diabetes patients.

In certain embodiments, chymosin-containing products may provide a treatment for migraine patients. Other embodiments are directed to chymosin-containing products being used as a replacement for lactose-free or lactose-reduced milk, preferably for lactose-intolerant consumers.

Preferably, chymosin can be added to pharmaceutical or food products as a prophylactic nutrition ingredient. In one embodiment, chymosin is added to a dairy product after pasteurization and after the milk has been chilled again; preferably, the dairy product is not heated again after the addition of chymosin. Preferably, the milk product is kept chilled before being administered so that the enzyme will not have any pulping effect before reaching the stomach. Preferably, chymosin is not activated until it reaches the stomach, where it is warmed to an effective temperature, normally to above 25° C.

In another embodiment, chymosin is given as a supplement directly to an infant before, during or after breast or bottle feeding.

In another embodiment, chymosin is administered just prior to feeding, such as preferably within about 30 minutes prior, within about 15 minutes prior and more preferably within about 5 minutes prior (including within about 4, 3, 2, and 1 minute prior), or it can be administered during feeding or mixed with nutritional supplements, foods and/or beverages. Compositions may also be administered shortly after feeding, such as, for example, preferably within about 30 minutes, more preferably within about 15 minutes after and even more preferably within about 5 minutes after feeding (including within about 4, 3, 2, and 1 minute after). Preferably, the compositions are designed for infants and contain a pharmaceutically acceptable carrier especially formulated for infants such as described in “Remington: The Science and Practice of Pharmacy 21^{st} Edition” (University of Sciences, Philadelphia ed.) (which is specifically and entirely incorporated by reference).

Administration is preferable at a pharmaceutically effective dose for the patient. Effective dose is determined empirically and calculated as described in Pharmaceutical Calculations 12^{th} Edition, by Howard C. Ansel, Mitchell J. Stoklosa (which is specifically and entirely incorporated by reference). In certain embodiments of the invention, calculations are typically based on body weight, making certain that there are no allergic reactions from the composition including all associated pharmaceutical carriers. Chymosin activity is typically measured in IMCUs (International Milk-Clotting Units). IMCU units are measured according to the measuring method described in ISO 15174/IDF 176. This equals GARNOT average enzyme activity (measuring method J.O. 20/03/1981). GARNOT number at 2,985 mg/l equals 200 IMCU/ml. In regular cheese production, the average use of chymosin is 35 IMCU per 1 liter of milk. Typically compositions in accordance with embodiments of this invention contain between 1 and 30,000 IMCU/ml, 1,100-30,000 IMCU/ml, or 1-1,100 IMCU/ml, more preferably 1-50 IMCU/ml, more preferably 1-100 IMCU/ml, more preferably 50-100 IMCU/ml, more preferably 100-200 IMCU/ml, more preferably 200-500 IMCU/ml, and more preferably 500-1,100 IMCU/ml. A therapeutically effective amount of certain embodiments of this invention is typically from 10-500 IMCU per liter of milk, more preferably 20-500 IMCU per liter of milk, more preferably from 10-200 IMCU per liter of milk, more preferably 20-200 IMCU per liter of milk, more preferably 10-100 IMCU per liter of milk, more preferably 20-100 IMCU per liter of milk, more preferably from 10-50 IMCU per liter of milk, more preferably 20-50 IMCU per liter of milk, but may be more or less depending upon, for example, how rapid a reaction is desired, the weight of the patient, or how firm a cheese pulp is desired.

The chymosin can be administrated as a liquid or as a dry solid. The chymosin can be added to drinking milk or another milk product, as soon as the milk has been cooled down following pasteurization. Pasteurization can destroy the chymosin, and other rennets. However, several types of rennets may be able to undergo the pasteurization process and remain intact. The chymosin can be added at any part of the production process in the dairy, and the product is most preferably maintained chilled afterwards.

The chymosin can also be added by the end user. It can be added as a liquid or as a dry solid, either directly to the milk or milk product, or it can be taken orally just before, during or immediately after a meal or consumption of milk. It can be administered or consumed as a separate drink, or as a liquid or tablet.

Chymosin is a proteolytic enzyme synthesized by chief cells in the stomach. Its role in digestion is to curdle or coagulate milk in the stomach. Absent chymosin, uncoagulated would rapidly flow through the stomach and miss the opportunity for initial digestion of its proteins. Chymosin efficiently converts liquid milk to a semisolid like cottage cheese, allowing it to be retained for longer periods of time in the stomach. Chymosin secretion is maximal during the first few days after birth, and declines thereafter, replaced in effect by secretion of pepsin as the major gastric protease.

Chymosin is secreted as an inactive proenzyme called prochymosin that, like pepsin, is activated on exposure to acid. Chymosin is also similar to pepsin in being most active in acidic environments. To understand how chymosin coagulates milk, it is helpful to understand milk proteins. Basically, the majority of milk protein is casein which has four major types of molecules: alpha-s1, alpha-s2, beta and kappa. The alpha and beta caseins are hydrophobic proteins that are readily precipitated by calcium—the normal calcium concentration in milk is far in excess of that required to precipitate these proteins. However, kappa casein is a distinctly different molecule in that it is not calcium-precipitable. As caseins are secreted, they self-associate into aggregates called micelles in which the alpha and beta caseins are kept from precipitating by their interactions with kappa casein. In essence, kappa casein normally maintains the majority of milk protein soluble and prevents it from spontaneously coagulating. Chymosin proteolytically cuts and inactivates kappa casein, converting it into para-kappa-casein and a smaller protein called macropeptide. Para-kappa-casein does not have the ability to stabilize the micellar structure, and the calcium-insoluble caseins precipitate, forming a curd. Aside from chymosin's physiologic role, chymosin is also a very important industrial enzyme because it is widely used in cheese making. Chymosin can be obtained from extracted dried calf stomachs, but preferably made recombinantly. Many proteases are able to coagulate milk by converting casein to paracasein and other alternatives to chymosin are commercially available.

In one embodiment, the enzymes of this invention are added to a dairy product or infant formula at the manufacturing stage; in another embodiment, the enzymes can be added at home by the user.

Preferably, chymosin is administered immediately before, during, or immediately after feeding; preferably, if needed, chymosin is administered at all three time intervals. All of the embodiments of this invention that have either been expressed or can be implied can also be practiced with other types of rennet, preferably a rennet designed for adding to drinking milk, in place of chymosin. Similarly, homologous proteins, aspartic proteases, and/or gastric proteases, preferably fetal or neonatal gastric proteases from vertebrates such as, for example, from pigs, lambs, buffalo, cows, goats chickens or humans, can be used in embodiments of this invention (Foltman, B., Chymosin: A short review on foetal and neo-natal gastric proteases, Scand J Clin Lab Invest 1992; 52 (Suppl. 210): 65-79; Awad, S. et al., Proteolytic Activities of Chymosin and Porcine Pepsin on Buffalo Cow, and Goat Whole and β-Casein Fractions, J. Agric. Food Chem. 1998, 46, 4997-5007).

Another embodiment is directed to compositions and methods of the invention that promote calcium absorption by the gastrointestinal tract of the patient, and a general improvement of digestion.

Compositions of the invention are added to food products such as, for example, milk and milk products. When added to milk, compositions may be added at the diary and preferably after pasteurization or other heat treatment. Other milk products include, but are not limited to yogurt, ice cream, butter, cheese and baking products. Again, compositions of the invention are preferably added after pasteurization or other heat treatment, but can be added at anytime after. The amount of chymosin or other enzyme incorporated can be easily determined by those skilled in the art from routine testing. Further, the amount my be tailored for adult, infant or

Another embodiment of the invention is directed to food products containing chymosin. The enzyme pepsin will have very much the same effect. Even if the effect is best in combination with milk, it can also be added to other products as beer, and various soft drinks. In a soft drink the chymosin will amplify the sweetness of sugar, with the result that there may be used less sugar in order to achieve the same sweetness. When used in soft drink or beer, chymosin is preferably added after any heat treatment.

The chymosin can also be added to drinking water, and mixed with other flavors if desired. Further the chymosin can also be added to fruit juices, and this can be combined with milk added to the fruit juice as well. Again the chymosin can be added to product any time after an eventual heat treatment. If milk is added to the fruit juice together with chymosin, it should be kept cooled. But if the milk is treated to be able to withstand ordinary room temperature, the chymosin will withstand it as well.

The chymosin may also be taken as a pill together with a meal, in order to improve digestion. One pill may contain from 10 to 1,100 IMCU.

In ice cream the chymosin may be mixed in either as a liquid, or as a powder. Either will be activated after it has been heated in the stomach.

When added to beer or soft drinks, product may be stored at room temperature. If the temperature should come much above 60 degrees centigrade during storage, the enzymes will be destroyed, and the affect disappear.

Any pills made from the dry powder, may also be stored at ordinary room temperature, but should be kept below 60 degrees centigrade.

Another embodiment is directed to the use of chymosin in cereal. The enzyme can be mixed with the cereal during production as dry powder. When the cereal is eaten together with milk, the chymosin will be activated in the stomach after it is heated up together with the milk and the acids in the stomach.

Another embodiment is directed to the use of chymosin in alcohol, which can used as a snaps for improved digestion. In addition, compositions can be easily flavored without interfering with the enzyme or nutritional function.

Another embodiment is directed to the use of chymosin in wine production. This will also improve the taste of the wine, together with improved digestion.

In some embodiments, treatment of patients comprises administration of a medicine that aides in the self-production of chymosin in the stomach.

The medicine containing chymosin is produced in a buffered liquid form and administered to infants in the form of approximately 1-3 ml containing 5-50 IMCU/ml, or more preferably 10-20 IMCU/ml just prior to every feeding. This method of administration is identical for both breast-feeding as well as formula-fed babies.

Compositions of the invention may also contain a further a bioactive agent for treating a disease or disorder. These additional bioactive agents may include antiviral, antibacterial, antifungal, antiparasitic, antimetabolic, antiglaucomic, anti-inflammatory or antineoplastic compounds, carbohydrates, amino acids, peptides, proteins, immunoglobulins, immunomodulators, dyes, toxins, enzymes, hormones, neurotransmitters, glycoproteins, radiolabels, radiopaque compounds, fluorescent compounds, cell receptor proteins, cell receptor ligands, mydriatic compounds, vasodilators, bronchodilators, local anesthetics, growth promoting agents, and/or regenerative agents and combinations thereof.

The following examples illustrate embodiments of the invention, but should not be viewed as limiting the scope of the invention.

### EXAMPLES

In order to understand more about the action of chymosin, and the influence of the consequential lower pH value in the stomach, the following tests were performed in the laboratory.

Four experiments were run on glasses of milk, each containing 200 ml of regular drinking milk with 2.6% fat content, pasteurized and homogenized, were heated to 37 degrees Celsius. Into these glasses were added different amounts of chymosin. The time until a firm cheese curd formed was recorded.

1. Into the first glass was added 1 drop of chymosin, equivalent to 42 IMCU per 1 liter of milk. After 30 minutes there was no significant reaction.
2. Into the second glass were added 2 drops of chymosin, equivalent to 84 IMCU per 1 liter of milk. After 12 minutes a firm curd had formed.
3. Into the third glass were added 3 drops of chymosin, equivalent to 126 IMCU per 1 liter of milk. After 8 minutes there was no significant reaction.
4. Into the fourth glass was added 1 drop of chymosin, equivalent to 42 IMCU per 1 liter of milk. In addition, 1.5 ml of concentrated hydrochloric acid (30%) was added. This lowered the pH value to 4.9. A firm curd had formed after 1 minute and 45 seconds.

Other embodiments and uses of the invention will be apparent to those skilled in the art from consideration of the specification and practice of the invention disclosed herein. All references cited herein, including all publications, U.S. and foreign patents and patent applications, are specifically and entirely incorporated by reference. The term comprising is used throughout the specification and it is intended that wherever used, the meaning includes the more limited terms consisting and consisting essentially of. It is intended that the specification and examples be considered exemplary only with the true scope and spirit of the invention indicated by the following claims.

1. A composition comprising a therapeutically effective amount of rennin formulated with an acceptable carrier for administration to a patient.

2. The composition of claim 1, wherein the therapeutically effective amount of rennin comprises 1-3,000 IMCU/ml.

3. The composition of claim 1, which is diluted before administration to the patient.

4. The composition of claim 1, wherein the rennin is pseudochymosin, prochymosin or chymosin.

5. The composition of claim 1, wherein the rennin is inactive.

6. The composition of claim 1, wherein the inactive rennin is subject to activation prior to or upon administration to the patient.

7. The composition of claim 1, wherein the rennin is isolated from mammalian tissue or isolated from microbes.

8. The composition of claim 1, wherein the rennin is recombinantly produced.

9. The composition of claim 1, wherein the acceptable carrier is a pharmaceutically acceptable carrier comprising water, alcohol, an acid, a base, glycerol, a saccharide or polysaccharide, an oil, a lipid, a buffer, a fatty acid, or a combination thereof.

10. The composition of claim 1, wherein the acceptable carrier is a nutritionally acceptable carrier comprising water, a food stuff, a drink, a nutritional supplement, or a combination thereof

11. The composition of claim 1, which further contains a stabilizer, a preservative, an anti-oxidant, a colorant, a flavoring agent or a combination thereof.

12. The composition of claim 1, wherein the patient is an infant, a toddler, a child, an adult or a senior.

13. The composition of claim 1, wherein the composition promotes calcium absorption by a gastrointestinal tract of the patient.

14. The composition of claim 1, wherein the composition enzymatically digests milk protein and coagulating milk inside a stomach after oral administration.

15. The composition of claim 1, which is a powder or a liquid form.

16. The composition of claim 1, wherein the composition is added to a food product.

17. The composition of claim 16, wherein the food product is milk or infant formula.

18. The composition of claim 1, wherein the composition prevents or ameliorates symptoms associated with the disorder selected from the group consisting of infant colic, heartburn, gastro-esophageal reflux, irritable bowel syndrome, digestive disorders, stomach disorders, bowel disorders, or combinations thereof recurrent abdominal pain, symptoms of osteoporosis, ulcerous colitis, multiple sclerosis, migraines and diabetes.

19. The composition of claim 1, wherein the composition promotes weight-loss.

20. The composition of claim 1, further comprising a bioactive agent for treating a disorder.

21. A method for producing the composition of claim 1.

22. A method for treating or preventing a disorder comprising:
administering a composition containing a therapeutically effective amount of rennin to a patient.

23. The method of claim 22, wherein the rennin is inactive when administered and is activated in the gastrointestinal tract of the patient.

24. The method of claim 22, wherein the rennin is activated by exposure to an increase in temperature or a decrease in pH value prior to administration to the patient.

25. The method of claim 22, wherein the disorder is selected from the group consisting of treating colic, heartburn, gastro-esophageal reflux, gastro-esophageal reflux disease, irritable bowel syndrome, recurrent abdominal pain, osteoporosis, ulcerous colitis symptoms, chronic digestion problems, multiple sclerosis, diabetes, obesity, weight problems, migraines, digestive disorders, stomach disorders, and bowel disorders.

26. The method of claim 22, wherein the composition is administered orally as an additive to a food product.

27. The method of claim 22, wherein the composition is administered to an infant before, during or after feeding.

28. The method of claim 22, wherein the composition is administered in a buffered liquid form comprising 5-50 IMCU/ml of active rennin.
